(19) United States
(12) Patent Application Publication
(10) Pub. No.: US 2009/0221932 A1
(43) Pub. Date: Sep. 3, 2009

(54) Microwave temperature image reconstruction

(76) Inventors: Torsten Butz, Niederteufen (CH); Emanuele Dati, Prilly (CH); Oscar Divorra Escoda, Princeton, NJ (US); Murat Kunt, Grandvaux (CH); Pierre Vandergheynst, Pully (CH)

Correspondence Address: Clifford W. Browning;Krieg DeVault, Suite 2800, One Indiana Square, Indianapolis, IN, 46204-2079, US

(21) Appl. No.: 11/988,067

(22) PCT Filed: Jun. 27, 2006
(86) PCT No.: PCT/IB2006/052129

§ 371 Date: Dec. 28, 2007

(30) Foreign Application Priority Data
Jun. 30, 2005 (EP) 05405418.4

(51) Int. Cl. A61B 5/01

(52) U.S. Cl. 600/549

(57) ABSTRACT

A multi-frequency microwave antenna (1) of the microwave image reconstruction system senses a signal which passes through an analogue connection (6) to a Dicke null-balancing radiometer (7) which detects sequentially the signal contributions at the different frequencies of the multi-frequency antenna of the system. The resulting sequential analogue signals from the radiometer are directly related to the brightness temperatures at the corresponding frequencies, and these brightness temperatures are directly related to a real intra-body temperature distribution. The system uses a PC (5) and comprises a mean to reconstruct a one-dimensional profile of real intra-body temperatures from the brightness temperatures via the outputs from the analogue-to-digital converter (15). The algorithm of this system uses “large” over-complete dictionaries which, on the one hand reflect the variability of potential temperature profiles and on the other hand incorporate as much a-priory information as possible, in order to provide reliable image reconstruction.

### TECHNICAL FIELD OF THE INVENTION

The present invention relates to a microwave temperature image reconstruction system. More specifically, the present invention relates to the use of an algorithm which shows itself particularly adapted to temperature image reconstruction.

### BACKGROUND OF MICROWAVE IMAGING

The large potential of temperature imaging has been recognized for long. For example in the medical community such data is known to potentially improve a variety of diagnostic and interventional procedures. In the case of industrial applications, thermal information has shown to predict and prevent failures of electrical circuits and equipment. One potentially interesting imaging modality for temperature imaging is microwave imaging. Due to several practical drawbacks, microwave imaging has hardly been able to enter the industrial or medical end-user market though. One of these drawbacks lies in the difficult image reconstruction task which contrasts with the rather simple data acquisition process of a potential microwave imaging system.

Non-Destructive Evaluation (NDE) has been one of the engineering disciplines which mostly revolutionized diagnostic techniques in industry and in medicine during the last decades. MR (Magnetic Resonance), CT (Computerized Tomography), US (Ultra-Sound), and other NDE devices are being standard tools for a wide range of diagnostic fields. Furthermore they are currently changing significantly medical surgery, as their capability of visualizing intra-body information enables surgeons to minimize the invasiveness of their interventions. Even though NDE techniques are by themselves expensive, they are potentially even interesting from a financial point of view, as they can significantly decrease the expensive hospitalization time of patients. Similar arguments apply to industrial NDE which was able to bring quality assurance and failure prediction to an impressive performance.

Several NDE modalities are currently being used, including MR, CT, and US imaging. Each of these modalities images a particular physical quantity, such as MR can image proton density, CT images x-ray absorption coefficients, or US visualizes reflection coefficients for ultrasound waves. Therefore each of these modalities has applications for which they are mostly adapted, while being excluded from other applications due to their incapability to image the appropriate physical quantity. As a result, the development of new NDE imaging modalities which are able to image additional physical quantities is a main branch of engineering and fundamental research. One of these quantities, which have been recognized to be particularly valuable for a wide range of medical and industrial applications, is intra-body temperature. Currently, just MR imaging is capable to perform such measurements non-invasively, though being prohibitively expensive for a wide range of promising applications. As an example, cancer development is known to be accompanied with a local temperature increase of 1-2° C. Nevertheless, the temperature resolution and mere cost of MR temperature imaging prohibits its use in the important field of breast cancer detection.

As an alternative to MR temperature imaging, passive microwave temperature monitoring is a promising avenue. Microwave temperature imaging devices are in comparison to MR devices significantly smaller, cheaper, and less complex to implement. Nevertheless, their current research implementations are too premature to be produced and marketed on large scales. One of the main drawbacks lies in the fact that the data collected by microwave imaging devices is very sparse (only around 7 independent measurements) in comparison to the temperature resolution and spatial resolution one would like to obtain for the final temperature profiles. This implies that the image reconstruction is particularly challenging for such devices.

### SUMMARY OF THE INVENTION

The present invention proposes a solution to this reconstruction problem. It uses “large” over-complete dictionaries which, on the one hand, reflect the variability of potential temperature profiles and, on the other hand, incorporate as much a-priory information as possible in order to provide reliable image reconstruction. Together with state-of-the-art algorithms for data decomposition using over-complete dictionaries, temperature profiles can be reconstructed reliably.

The system according to the invention uses passive microwave imaging for temperature imaging, where “passive” refers to the fact that the imaging system does not emit any radiation into the investigated object. Rather, it senses and measures the natural thermal radiation emitted from this object, which contrasts to active and scattering-based microwave imaging devices.

The above and other objects, features and advantages of the present invention will become apparent from the following description when taken in conjunction with the accompanying drawings which illustrate preferred embodiments of the present invention by way of example.

### BRIEF DESCRIPTION OF THE DRAWINGS

FIG. 1 shows 3 B-splines of a set of 100 B-splines which cover regularly the imaging space and whose weighted sum constitutes the temperature profile reconstruction.

FIG. 2 shows three results of current state-of-the-art microwave temperature profile reconstruction by Tikhonov regularization and its limitations. The reference and reconstructed profiles are shown on the left side, while the resulting expansion coefficients are presented in the right column.

FIG. 3 shows the basic building blocks necessary for a microwave temperature monitoring device with a reconstruction procedure as the one disclosed herein.

FIG. 4 shows a schematic sketch of the functional building blocks necessary to realize the disclosed invention.

FIG. 5 shows the functional building blocks for data decomposition with over-complete dictionaries as used in the disclosed invention.

FIG. 6 presents a sketch of the building blocks for a PC-based implementation of the disclosed invention.

FIG. 7 shows a more detailed view of the PC-based sample implementation of a microwave temperature imaging device.

FIG. 8 shows cubic B-splines at scale 0 and scale 9 covering regularly the imaging space. These functions were part of the over-complete basis function set, used in the sample implementations of the disclosed invention.

FIG. 9 presents three reconstruction examples for the PC-based sample device. On the left side, the reconstructed and reference profiles are drawn. On the right side, the determined optimal expansion coefficients are shown. These coefficients correspond to the reconstruction profiles shown on the left.

FIG. 10 shows a sketch of the functional building blocks for the embedded sample implementation of the disclosed invention.

FIG. 11 shows a more detailed view of the embedded sample implementation of a microwave temperature imaging device.

FIG. 12 shows the flow chart of the FOCUSS algorithm as used for the temperature profile image reconstruction on the embedded sample implementation of the disclosed invention.

FIG. 13 shows three reconstruction results for the embedded sample device. The reconstructed and reference profiles are drawn on the left side, while the optimal expansion coefficients are drawn on the right side.

### DETAILED DESCRIPTION OF THE INVENTION

Passive microwave image reconstruction aims to solve the following equation for the unknown temperature profile, noted T(x):

$${b}_{i} = {γ}_{i} \cdot {\int }_{Ω} {W}_{i} ( x ) \cdot T ( x ) d x , \\ with \\ i = 1 , 2 , … , M , \qquad Eq . 1$$

where i indexes the M measurements of the system at frequencies fᵢ. bᵢ are the passively measured brightness temperatures sensed by the multi-frequency imaging antenna at different frequencies fᵢ, as described in [2, 9]. γᵢ are the antenna efficiencies at frequencies fᵢ, Wᵢ(x) are the spatial weighting functions at frequencies fᵢ, and Ω is the sensing space of the microwave antenna. The spatial variable x may be a 1D, 2D, or 3D spatial vector, depending on the dimension of the reconstruction problem. In the 1D case (just one multi-frequency antenna), the number of measurements M lies at around 5 to 7, i.e. the antenna senses the brightness temperatures bᵢ at 5 to 7 different frequencies fᵢ. Therefore we have just 5 to 7 measurements to recover the whole 1D temperature profile T(x).

The weighting function Wᵢ(x) weights the brightness temperature contributions from different spatial positions x. It therefore accounts for the absorption and scattering process of the temperature dependent thermal radiation from any point inside the investigated body to the passive measurement antenna.

Algorithms currently being used for the microwave image reconstruction of eq. 1 are presented in [1, 2]. Both approaches attempt to counter-balance the problem of having just few radiometric measurements available for the reconstruction, by giving a very restrictive prior on the temperature profile shape one would like to recover. In [1] and in the case of a 1D profile, the profile is assumed to be a single Gaussian:

$$T ( x ) = {T}_{0} \cdot exp ( - ( \frac{x - {x}_{0}}{σ} ) ) , \qquad Eq . 2$$

with T₀ being the amplitude of the Gaussian, x₀ its position and σ its variance. The reconstruction algorithm determines the parameters, T₀, x₀ and σ, which explain optimally the measurements bᵢ through eq. 1. The problem with this approach lies in the very strong Gaussian assumption of eq. 2. This assumption is too restrictive to deal with a large number of real world temperature profiles.

A more flexible approach to temperature image reconstruction has been proposed in [2]. There, a small sum of Chebyshev polynomials is assumed to build the temperature profile T(x):

$$T ( x ) = {\sum }_{k = 1}^{K} {t}_{k} \cdot {h}_{k} ( x ) , \qquad Eq . 3$$

where the hₖ(x) are the Chebyshev polynomials, and the tₖ are the respective weighting coefficients. K is the number of polynomials in the sum which is assumed to result in a good approximation of the real temperature profile T(x) by eq. 3. Using eq. 3, eq. 1 can be re-written in matrix form:

b=A·t, Eq. 4

with

$${A}_{i , k} = {γ}_{i} \cdot {\int }_{Ω}^{ } {W}_{i} ( x ) \cdot {h}_{k} ( x ) d x .$$

This notation leads quite naturally to matrix inversion and Tikhonov regularization as used in [2] to solve the microwave reconstruction problem with Chebychev polynomials or potentially other functions. This approach though has still some important shortcomings which will be illuminated in the following paragraphs, and which motivated the disclosed invention.

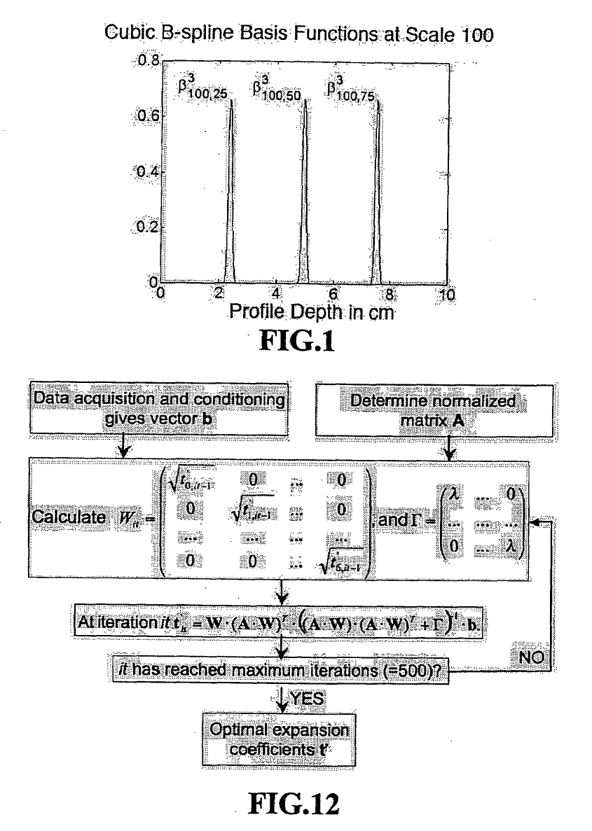

Let us shortly assume an example where the Chebychev polynomials of [2] are replaced by 100 B-splines of order 3 (cubic B-splines) at one single scale and covering the whole imaging depth. The sum of eq. 3 with 100 B-splines would be able to represent a very large variety of temperature profiles T(x). In FIG. 1, three of the 100 B-splines are drawn. Using all these 100 basis functions to reconstruct from the, let's say, 7 microwave measurements a temperature profile is not trivial, as one would try to solve a system of 7 equations for 100 unknowns tₖ (eq. 4 with A being a 7×100 matrix, i.e. an ill-posed problem). Such a system is heavily underdetermined, which means that a large or probably even infinite number of solutions exists. In [2], Tikhonov regularization is proposed in order to deal with the ill-posed character of the resulting reconstruction problem. It applies a smoothness constrained by imposing a minimal l₂-norm on the coefficients t, i.e. solving the following minimization problem in the least-square sense for t:

min∥A·t−b∥+λ·t·t^{T}, Eq. 5

where λ weights the importance of the smoothing constraint when solving eq. 1 for the optimal temperature profile, T(x). On the contrary to eq. 4, eq. 5 is not an ill-posed problem, as long as λ stays sufficiently big. The least-square solution to eq. 4 is written:

t=(A^{T}·A+λ·I)⁻¹·A^{T}·b. Eq. 6

Here, I is the identity matrix. One can see that when λ gets too small and the matrix A^{T}·A is ill-conditioned (which is in general the case for microwave temperature imaging) the inverse in eq. 6 does not exist.

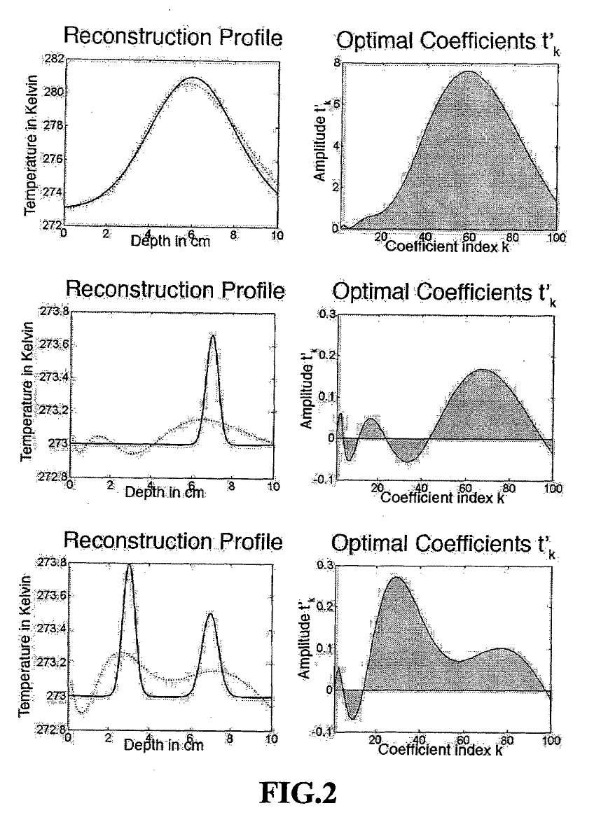

In FIG. 2, we show some reconstruction results using the algorithm of [2] with 100 B-splines at one scale covering regularly the imaging space and which do not correspond to an over-complete set of basis functions. The reconstruction results are represented in broken lines and the references in full lines. One sees easily that this approach does not always lead to satisfactory results. The regularization term over-smoothes the reconstruction result when the temperature profile to be recovered is not smooth enough itself. On the other, it's not possible to decrease the smoothing parameter, λ, beyond some minimum, as this would result again in inverting an ill-conditioned matrix A^{T}·A+λ·I. On the right hand side of FIG. 2, one can also observe that this state-of-the-art approach to profile reconstruction results in a very large weighted sum of B-spline functions, as almost all K coefficients tₖ are significantly larger than zero. In other words, Thikhonov regularization is neither appropriate for the described microwave image reconstruction problem. These arguments describe the motivations for the disclosed invention. They can be summarized as follows:
1) The reconstruction algorithm has to deal with the ill-posed character of the reconstruction problem, while being applicable to a wide range of possible real-world temperature profiles.
2) The algorithm must not be dominated by any regularization term provoking over-smoothing or other artifacts to appear.

The general solution to these problems as disclosed in this invention can be summarized as follows:
1) A large number K of basis functions, building an over-complete dictionary, has to be used in order to represent the variety of possible real-world temperature profiles with a small number N of these basis functions. As an example, the basis functions might be B-splines at different scales and positions. B-splines at several scales result in an over-complete set of basis functions. The fact that a small sum of N basis functions (small in the sense of N<<K) is sufficient for the reconstruction avoids over-smoothing of the resulting temperature profile estimation.
2) A regularized reconstruction algorithm has to be used which selects the optimal set of N basis functions out of the over-complete set of K initial basis functions (the over-complete dictionary) and which determines their weights to reconstruct the temperature profile. The regularization has to be as not to over-smooth the reconstruction result as shown to happen with e.g. Tikhonov regularization.

In conclusion, the disclosed invention describes a temperature image reconstruction procedure which, on the one hand, is capable to deal with the sparseness of the acquired radiometric data (just around M=7 measurements), and, on the other hand, succeeds in reconstructing a wide range of real-world temperature profiles for real-world medical and industrial applications without artifacts from the regularization term.

As will be shown in more detail in the following paragraphs, the disclosed invention maps the reconstruction problem into a well known formulation of decomposing signals with over-complete dictionaries and uses standard algorithms, such as matching pursuit [3], orthogonal matching pursuit [4], basis pursuit [5], high-resolution matching pursuit [6], etc. to solve the resulting equivalent problem.

The disclosed invention applies equivalently to 1D (profiles), 2D (images), or 3D (volumes) temperature image reconstruction.

### Detailed Description of the Preferred Embodiments

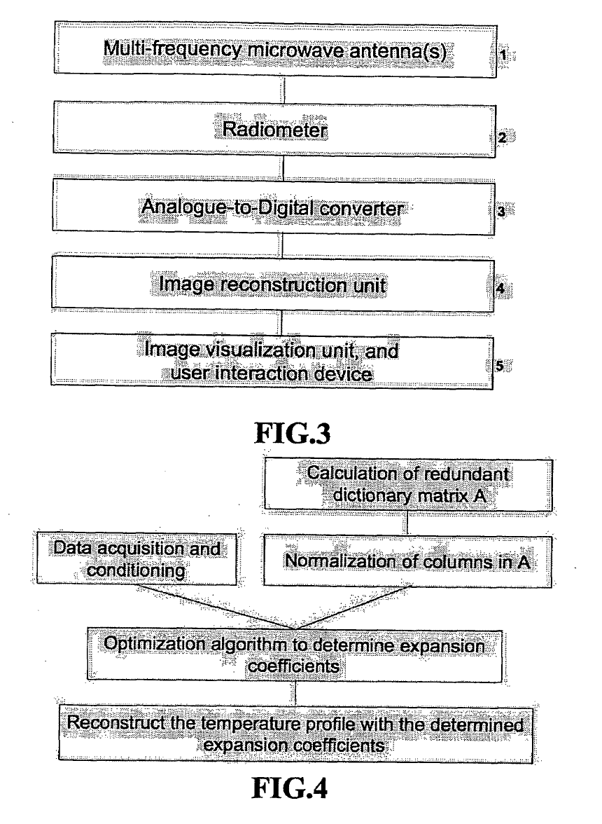

The disclosed invention for radiometric temperature image reconstruction has to be implemented on a completely functional microwave temperature imaging device. Several design implementations of such a system are possible, all of which share common characteristics though. In FIG. 3, the functional building blocks common to all these systems are presented. First, we need one or several passive multi-frequency microwave antennas 1 for sensing the brightness temperatures, next a radiometer 2 has to interpret the signal and translate the sensed microwave radiation power into units of voltage. An analogue-to-digital converter 3 then digitizes the analogue voltage, so that the reconstruction unit 4, basically any digital signal processing device, can reconstruct the intra-body temperature profile which caused the sensed microwave radiation. As a final step, the temperature profiles are visualized for inspection by the medical doctor or industrial investigator on a visualization unit, such as a computer screen 5. The disclosed invention is physically situated in the reconstruction unit 4 and describes how temperature profiles can reliably be reconstructed from the sparse microwave radiation measurements. This reconstruction procedure will be described next.

Microwave image reconstruction aims to solve the following integral equation:

$$b ( f ) = {γ}_{f} \cdot {\int }_{Ω}^{ } W ( f , x ) \cdot T ( x ) d x , \qquad Eq . 7$$

where b(f) is the measured brightness temperature sensed by the passive imaging antenna at frequency f, γ_{f} is the antenna efficiency at frequency f, W(f,x) is the spatial weighting function at frequency f, Ω is the sensing space of the microwave antenna, and T(x) is the spatial temperature distribution the reconstruction algorithms aim to recover. The weighting functions W(f,x) might be obtained from a analytical model, as presented in [2] for the 1D case, or by electromagnetic simulations. The spatial variable x may be a 1D, 2D, or 3D vector, depending on the dimension of the reconstruction problem. Eq. 1 is known as a Fredholm equation of the first kind. There are several approaches to solving such equations [7]. In the concrete case of microwave temperature imaging, eq. 7 takes a slightly different form though. This is due to the fact that the brightness temperatures b(f) can practically only be measured at a very restricted number of discrete frequencies, noted fᵢ, i=1, 2, . . . , M, with M around 5 to 7 for the 1D case. This small number of measurements, referred to as sparse data, stands in contrast to the resolution of the temperature profile T(x) one aims to obtain. Therefore, it is practically impossible to discretize T(x) to T(xⱼ), j=1, 2, . . . , P, where the xⱼ are the pixel coordinates, in order to find the matrix expression of eq. 1 as b=W·t, and using standard quadrature theory for the solution of eq. 1, such as presented in [8]. This is because in the simplest 1D case, the number P would have already to lie at around 256 or 512 (number of pixels along the 1D profile) in order to reach a practically valuable resolution for the temperature profile T(x). In other words, the matrix formulation would result in a system of only 5 to 7 linear equations with 256 or 512 unknowns T(xⱼ). Obviously this is a rather hard, if not impossible task to be done reliably, and in the case of a 2D or even 3D reconstruction, this problem would be become even harder.

Alternatively, and in contrast to the classical approach to solving Fredholm equations of the first kind, we reformulate eq. 7 in a semi-discrete fashion as follows:

$${b}_{i } = {γ}_{i} {\int }_{Ω}^{ } {W}_{i} ( x ) \cdot T ( x ) d x , with i = 1 , 2 , … , M \qquad Eq . 8$$

where i indexes the M discrete measurement frequencies fᵢ of the system. This formally quite simple change in problem formulation gives the possibility to solve the described image reconstruction problem for microwave radiometry in an attractive way, in particular for cases where the number M of equations in eq. 8 is small in comparison to the resolution one would like to obtain for the temperature profile T(x). In the following this approach is described in detail.

Intuitively, in order to solve eq. 8 for the unknown function T(x), one has to attempt to find a representation for the space of possible temperature profiles T(x), which has to be entirely determined by not much more than the number M of frequencies at which measurements are being carried out, while reflecting the large variability we might find in real-world temperature profiles. In addition, we have to propose algorithms which determine the optimal fit, i.e. the most probable function T(x) for the given measurements bᵢ. The disclosed reconstruction procedure exactly presents a general approach to solve such a problem for microwave radiometry. In general, the disclosed approach to solve eq. 8 for T(x) can be structured into the following two steps:
1. Construct a function sub-space of L₂ (space of square-integratable functions), denoted Ω_{D}, spanned by an over-complete set of basis functions, D={hₖ(x)}, k=1, 2, . . . , K, which accounts for the variability of possible temperature profiles T(x). The overcompleteness of the basis functions {hₖ(x)} means that for any function f(x) from Ω_{D}, L>1 sets {t_{k,i}}, i=1, 2, . . . , L of coefficients exist for which

$$f ( x ) \approx {\sum }_{k = 1}^{K} {t}_{k , i} \cdot {h}_{k} ( x ) , i = 1 , 2 , … , L . \qquad Eq . 9$$

In addition, the set {hₖ(x)} has to be constructed in a way that for any temperature profile T(x), which can realistically be expected, one set of parameters {tₖ} out of the L sets of parameters {t_{k,i}}, i=1, 2, . . . , L exists, which approximates the profile T(x) according to eq. 9 with just N<<K non-zero coefficients:

$$T ( x ) \approx {\sum }_{k = 1}^{K} {t}_{k} \cdot {h}_{k} ( x ) , \qquad Eq . 10$$

with the majority of the coefficients tₖ being zero, and just about N of the K coefficients having significant magnitude (N<<K).
2. Once such a set D has been formed, for any given set of measurements bᵢ an algorithm has to determine both, the N functions of D and its non-zero weighting coefficients tₖ which approximate the temperature profile T(x) optimally by the small weighted sum given in eq. 10.

The next paragraphs describe these two general steps in more detail.

### 1. Over-Complete Set of Basis Functions for Microwave Radiometry

Theoretically, any set of functions of L₂ would be admissible to span the reconstruction subspace of L₂, noted Ω_{D}, and therefore constitute the basis set, D. Practically though, any prior information about the temperature profile T(x) that can be incorporated into the chosen function set D should be employed in order to be able to implement an efficient and precise reconstruction algorithm. Furthermore, the basis set should be constructed, so that for any real-world temperature profile T(x) just a small subset of N basis functions from the potentially very large set of K basis functions (small in the sense of N<<K) is able to reconstruct T(x). Mathematically this means that real-world temperature profiles T(x) should have sparse representations in the space Ω_{D}.

Once the function set D has been constructed, one has still to implement an algorithm which is able to determine the small set of N basis functions from the initial over-complete dictionary of K functions, and its associated non-zero weights tₖ which reconstruct an estimate of the unknown temperature profile T(x):

$$T ( x ) \approx {\sum }_{k = 1}^{K} {t}_{k} \cdot {h}_{k} ( x ) , \qquad Eq . 11$$

with just N<<K non-zero coefficients tₖ. In other words, this algorithms has to determine the set of coefficients {tₖ} from the sets of coefficients {t_{k,i}}, i=1, 2, . . . , L of equation 9 for which most coefficients are zero.

In mathematical terms, we need an algorithm which determines a sparse representation of the temperature profile T(x) characterized by the set of measurements bᵢ within the subspace Ω_{D} of L₂, spanned by the set of functions D. In the next paragraph such algorithms are shown

### 2. Reconstruction Algorithm

The initial reconstruction problem of eq. 7 can be rewritten, using the reconstruction approach described in the previous paragraph. Introducing eq. 11 into eq. 8, we get:

$${b}_{i} = {\sum }_{k = 1}^{K} {t}_{k} \cdot {γ}_{i} \cdot {\int }_{Ω}^{ } {W}_{i} ( x ) \cdot {h}_{k} ( x ) d x . \qquad Eq . 12$$

This problem formulation can also be written in matrix form as:

b=A·t, Eq. 13

with

$${A}_{i , k} = {γ}_{i} \cdot {\int }_{Ω}^{ } {W}_{i} ( x ) \cdot {h}_{k} ( x ) d x . \qquad Eq . 14$$

The size of the matrix A is given by the number of measurements M of the system, and by the number of functions K in the over-complete set D. The number of rows is therefore M, while the number of columns is K.

Eqs. 12 and 13 allow reinterpreting the temperature image reconstruction in a way which gives direct access to a large range of algorithms perfectly adapted to the problem of finding the optimal weighting coefficients tₖ for the profile reconstruction through eq. 10. In fact, eq. 14 can be seen as a transformation F, which transforms the space Ω_{D} spanned by the basis functions of the set D onto a subspace of R^{N} (the vector space of N-dimensional vectors of real numbers), denoted Ω_{R}:

$$F : {Ω}_{D} ⋐ {L}_{2} \to {Ω}_{R} ⊆ {R}^{N} , \\ {h}_{k} ( x ) \to {a}_{k} = {\int }_{Ω}^{ } W ( x ) \cdot {h}_{k} ( x ) d x , \\ k = 1 , 2 , … , K , \\ with \\ W ( x ) := ( \begin{matrix}{γ}_{1} \cdot {W}_{1} ( x ) \\ {γ}_{2} \cdot {W}_{2} ( x ) \\ KK \\ {γ}_{M} \cdot {W}_{M} ( x )\end{matrix} ) \qquad Eq . 15$$

The resulting subspace Ω_{R} of R^{N} can be seen as a vector space spanned by the vectors {aₖ} which are just the rows in the matrix A of eq. 13. As a last step, we normalize the vectors aₖ and therefore the rows of A and reformulate the temperature profile reconstruction of eq. 14 accordingly:

$$b = {\sum }_{k = 1}^{K} {t}_{k}^{′} \cdot {a}_{k}^{′} , \\ with \\ {t}_{k}^{′} = {t}_{k} \cdot ‖ {a}_{k} ‖ , \\ and \\ {a}_{k}^{′} = \frac{{a}_{k}}{‖ {a}_{k} ‖} . \qquad Eq . 16$$

The symbol ∥aₖ∥ stands for the classical vector norm of R^{N} defined by

$$‖ {a}_{k} ‖ = \sqrt{{\sum }_{i} {a}_{i}^{2}} .$$

Eq. 16 is the reformulation of the initial temperature profile reconstruction, characterized by eq. 7, as standard sparse data decomposition using over-complete dictionaries. Solving eq. 17 for a sparse representation aims to determine the few non-zero expansion coefficients t′ₖ, which result in a good approximation of b. Matching pursuit [3], orthogonal matching pursuit [4], basis pursuit [5], high-resolution matching pursuit [6], etc. are known algorithms for this task. Once this decomposition of b has been determined, the one-to-one correspondence of eq. 15 between the over-complete set of base vectors {a′ₖ} of Ω_{R} and the over-complete set of basis functions {hₖ(x)} of Ω_{D} enables the temperature profile reconstruction through eq. 10:

$$T ( x ) \approx {\sum }_{k = 1}^{K} \frac{{t}_{k}^{′}}{‖ {a}_{k} ‖} \cdot {h}_{k} ( x ) . \qquad Eq . 17$$

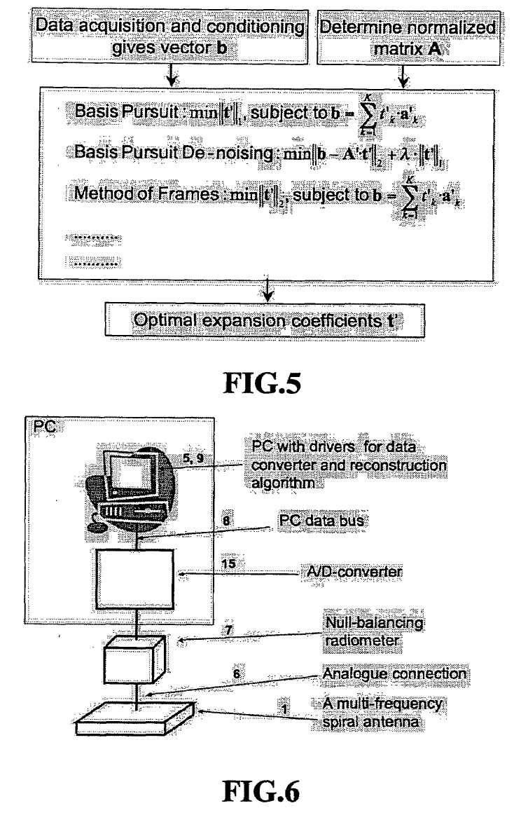

In FIG. 4, a functional block-diagram is shown, which describes the different tasks that have to be implemented for the disclosed invention. In FIG. 5, some algorithms that can be used in this disclosed invention are shown in relationship to its surrounding functional blocks. The mentioned algorithms are not the only possible algorithms which would solve eq. 16. Common to all of them is that they consist of two parts. First, they have the data part, which considers the actual measurements to determine the optimal temperature profile T(x). And second, there is a sparseness constraint which ensures that just a small number N (small in the sense of N<<K) of the coefficients t′ₖ in eq. 17 has significant amplitude (sparse representation) and which, in the case of microwave temperature image reconstruction, plays the role of a regularization term that turns the generally ill-posed inversion problem of microwave temperature image reconstruction into a well-posed inversion problem. The main difference of these “sparseness regularization terms” to the smoothness constraint of Tikhonov regularization (eq. 5), is that they do not over-smooth the final reconstruction result T(x). Let's shortly describe some of these well-known algorithms.

In the notational context of the previous paragraphs, general sparse decomposition using over-complete dictionaries tries to solve the following equation:

min|t′∥₀, subject to b=A′·t′, Eq. 18

where ∥.∥₀ denotes the l₀-norm. The minimization of the l₀-norm results in searching for a sparse representation, as the l₀-norm counts the non-zero expansion coefficients t′ₖ in the expansion and tries to minimize their number N under the constrained that the data term is valid. Unfortunately, this problem can only be solved in a combinatorial way, and is therefore computationally very expensive. Therefore one approach consists of replacing the l₀-norm with a l₁-norm:

min∥t′∥₁, subject to b=A′·t′. Eq. 19

where ∥.∥₁ denotes the l₁-norm. The fact of minimizing the l₁-norm of t′ results in searching for a sparse representation, i.e. just few expansion coefficients t′ₖ have significant magnitude. On the contrary to eq. 18, this problem can be solved with linear programming [5]. Interior point methods or simplex methods [13, 14, 15] can be used to solve eq. 19. Matlab's Optimization Toolbox [12, 15] has standard implementations of these algorithms which are perfectly suited to solve eq. 19.

A similar approach as basis pursuit is called basis pursuit denoising. It solves the following equation:

min|b−A′·t′∥₂+λt′λ₁, Eq. 20

where λ is a weighting coefficient, weighting the importance of the regularization term based on the l₁-norm of the decomposition coefficients t′ₖ, and ∥.∥₂ denotes the l₂-norm. The l₁-norm component of the minimization problem results in searching for a sparse representation of the initial reconstruction problem. This formulation of sparse data decomposition using over-complete dictionaries can use quadratic programming [16, 17]. Again, this is computationally much more efficient than trying to solve eq. 18, and as for eq. 19, there is a standard implementation of quadratic optimization in Matlab's Optimization Toolbox [12].

As mentioned, a large number of further known algorithms exist, which can be applied to the presented invention, i.e. to sparse microwave temperature image reconstruction using over-complete dictionaries. A complete overview of these algorithms would go beyond the scope of this text though. Common to all these algorithms is there localization within a concrete system design which would implement the disclosed invention, as was outlined in FIGS. 4 and 5.

It has to be noted that Thikonov regularization as presented in [2] is not part of the presented class of algorithms, as it does not search for a sparse solution of the inversion problem. This is shown in FIG. 2, where almost all coefficients t′ₖ have significiant magnitude. In addition, the employed set of Chebychev polynomials does not form a large and over-complete set of basis functions.

### Detailed Description of Further Embodiments

As mentioned, the described reconstruction approach is applicable to 1D, 2D, or 3D reconstruction. The example implementations presented herein are in the context of the reconstruction of 1D temperature profiles for medical applications. Several medical applications could potentially take important profit from an easy-to-use and relatively cheap temperature measuring system. For example, thermal tumor ablation is a medical procedure for cancer treatment whose success is directly related to the exact control of the intra-body temperature distribution. During a thermal tumor ablation treatment, thin needles are introduced percutaneously until their tips reach inside the tumor volume. At the tips, radio-frequency heats the tumor tissue up to a temperature guaranteeing cell death. In this procedure, two characteristics are fundamental for the success of the treatment: On the one hand, all the cancerous tissue has to be killed, and on the other hand, as few healthy tissue as possible should be ablated. As in such a treatment, the killing of the body cells is directly related to reaching or not reaching a particular temperature threshold, the specific system implementation presented herein can significantly improve the security and reliability of the medical treatment plan through real-time monitoring of the internal temperature distribution.

Tumor detection is mentioned as a second field of medical application for the presented system implementation. It employs the fact that tumor cells manifest themselves through a small local temperature increase of 1-2° C. Therefore, the temperature monitoring capability of the presented system allows the medical doctor to use temperature as an additional manifestation of cancerous cells during cancer screening.

Any concrete implementation of a microwave temperature imaging device has to contain a certain set of functional building blocks. These building blocks were sketched schematically in FIG. 3. In the following, two concrete sample implementations of the general functional system are presented. Their main differences lie in the signal processing unit on which the images are reconstructed, and on the concrete reconstruction algorithm being employed for the reconstruction. Concretely, the first sample implementation uses an off-the-shelf personal computer (PC) as the signal processing unit for the image reconstruction, and the employed algorithm is a Matlab implementation of the basis pursuit algorithm as presented in [5]. The second implementation uses an embedded hardware device and the FOCUSS algorithm is used to find the basis pursuit de-noising solution to the reconstruction problem as presented in [18].

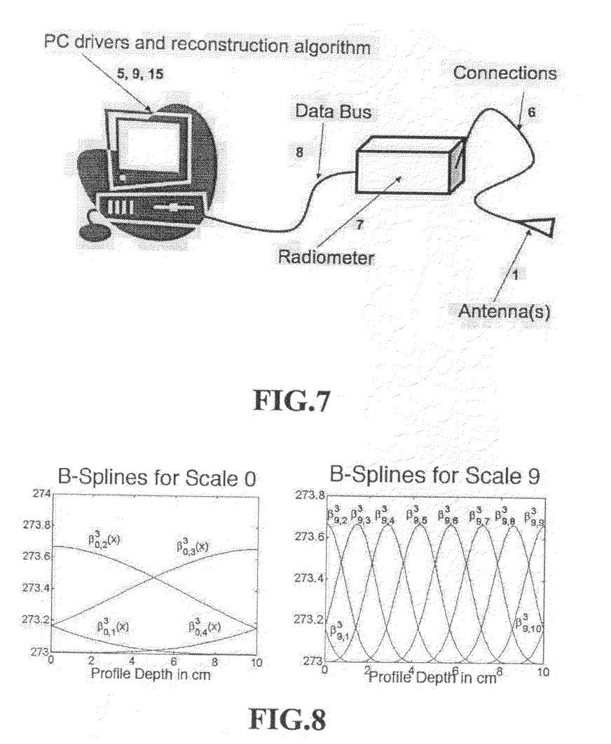

FIGS. 6 and 7 illustrate a system comprising a multi-frequency microwave spiral antenna(s) or wave guide antenna(s) 1, an analogue connection or connections 6, a Dicke-null-balancing radiometer 7, a PC data bus, such as USB, PCI, FireWire, or others 8 and a PC. The system according to FIG. 6 comprises an off-the-shelf-PC 5, 9 with Matlab drivers for the analogue-to-digital data converter (Iotech DaqBoard/2000 Series) 15 and with the reconstruction algorithm implemented in Matlab. The system according to FIG. 7 comprises a PC 5, 9, 15 with Iotech DaqBoard/2000 Series, corresponding Matlab drivers and reconstruction algorithms implemented in Matlab. In FIGS. 6 and 7, the building blocks of the PC-based sample implementation are schematically shown, while the embedded sample implementation is drawn in FIGS. 11 and 12. Before presenting the implementation specific aspects of the devices, the common parts of both implementations are presented.

Both systems comprise one single (1D temperature profile) multi-frequency spiral microwave antenna 1 as disclosed by Jacobsen and Stauffer in [9]. The employed antenna operates at 7 frequencies in the range of 0.5 to 3.75 GHz. The signal sensed by the antenna passes through an analogue connection 6 to a Dicke null-balancing radiometer 7, as described by Jacobsen and al. in [10]. The Dicke radiometer detects sequentially the signal contributions at the different frequencies of the multi-frequency antenna of the system. The resulting sequential analogue signals from the radiometer are directly related to the brightness temperatures at the corresponding frequencies, and these brightness temperatures are directly related to the real intra-body temperature distribution through eq. 8. In order to reconstruct a one dimensional profile of intra-body temperatures from the brightness temperatures, the reconstruction procedure disclosed herein is getting applied to the brightness temperatures, i.e. to the outputs from the analogue-to-digital converter 3, 10, 15.

As mentioned in the general description, any specific reconstruction implementation needs to specify two aspects: first, the over-complete set of basis functions {hₖ(x)} has to be specified, and second, the optimization algorithm has to be chosen. It is important to note that in the present context of 1D temperature profile reconstruction, the spatial variable x parameterizes just a 1D space Ω, i.e. is represented by one-component vectors. The size of the imaging space Ω is given by the maximal sensing depth dₘₐₓ of the microwave antenna, such as e.g. 10 cm for the cases presented herein.

In real-world medical applications for microwave temperature monitoring, such as cancer detection or thermal tumor ablation monitoring, bell-shaped temperature distributions can be expected. Therefore, we propose to use 1D B-splines at different scales and positions to build up the over-complete set D of K basis functions.

Herein, B-splines of order N_{d}=3 (cubic B-splines) and at Nₛ=30 consecutive scales are used to build the over-complete set D of base functions. The overcompleteness of D arises from the different scales of the B-splines. At the lowest scale, 4 B-splines cover the whole reconstruction space Ω, while at each higher scale, one spline is added. Therefore, at each scale s, Nₚ₍ₛ₎=s+4 B-splines at different positions are added to the set D, and the whole over-complete set of basis functions can be parameterized as:

D={hₖ(x)}={β_{s,p}(s)^{N}^{_{d}}(x)}, Eq. 21

### N_{d}=3,

s=0, 1, . . . , 29,
p(s)=1, 2, . . . , s+4.
with

$${β}_{s , p ( s )}^{3} ( x ) = \{ \begin{matrix}\frac{2}{3} - {\vert x \vert}^{2} + \frac{{\vert x \vert}^{3}}{2} , & if 0 \leq \vert \begin{matrix}( x - {d}_{max} \cdot \frac{( p ( s ) - 2 )}{{N}_{p ( s )} - 3} ) \cdot \\ \frac{{N}_{p ( s )} - 3}{{d}_{max}}\end{matrix} \vert \leq 1 , \\ \frac{{( 2 - \vert x \vert )}^{3}}{6} , & if 1 \leq \vert \begin{matrix}( x - {d}_{max} \cdot \frac{( p ( s ) - 2 )}{{N}_{p ( s )} - 3} ) \cdot \\ \frac{{N}_{p ( s )} - 3}{{d}_{max}}\end{matrix} \vert \leq 2 , \\ 0 , & otherwise .\end{matrix} \qquad Eq . 22$$

In FIG. 8, the cubic B-spline basis functions for scale 0 and 9 are drawn in dependence on the profile depth in cm. The cardinality of D, |D|=K, is given by:

$$K = \vert D \vert = {\sum }_{s = 0}^{{N}_{s} - 1} {N}_{p ( s )} = {\sum }_{s = 0}^{{N}_{s} - 1} s + 4 \qquad Eq . 23$$

Therefore in the present case, we have |D|K=555. In accordance with the disclosed reconstruction procedure, we have to transform the base functions of D by the transformation given in eq. 15. In order to be able to do this, we need to specify the explicit weighting functions characterizing the used antenna. In the case of 1D reconstruction, an analytic model of the weighting function is given in [2]:

$${W}_{i} ( x ) = \frac{1}{{d}_{i}} \cdot {e}^{- x / {d}_{i}} , \qquad Eq . 24$$

where the parameters dᵢ are called the antenna sensing depths at frequencies fᵢ. For the employed spiral antenna and the chosen frequencies, the sensing depths lie at {0.9 cm, 1.2 cm, 1.5 cm, 1.8 cm, 2.1 cm, 2.4 cm, and 2.7 cm}. For this weighting function and for the chosen B-spline basis functions, an analytical solution for the transformation integral of eq. 15 exists, which can be determined easily using the following recursive formula:

$$\int {x}^{n} \cdot {e}^{ax} d x = \frac{1}{a} \cdot {x}^{n} \cdot {e}^{ax} - \frac{n}{a} \int {x}^{n - 1} \cdot {e}^{ax} d x . \qquad Eq . 25$$

This equation can be verified easily using partial integration, or it can be found in mathematical reference books such as in [23]. Therefore the vectors {aₖ′} can be calculated analytically. If the transformation integral of eq. 15 cannot be solved analytically because the weighting functions Wᵢ(x) have a more complex form than given in eq. 24, or because electromagnetic simulations have been used to obtain a discrete model of these weighting functions, numerical integration has to be employed.

The reconstruction process can formally be summarized for the sample implementations presented in this section as follows:

b=A′·t′, Eq. 26

where b is the vector whose components are the measured brightness temperatures at the 7 frequencies. The matrix A′ is constituted of K=555 normalized vector columns with 7 components each, noted aₖ′, and t′ are the K=555 expansion coefficients one likes to recover. Therefore, eq. 14 becomes explicitely:

$${A}_{i , k}^{′} = \frac{{A}_{i , k}}{‖ {a}_{k} ‖} , \\ with \\ {A}_{i , k} = {\int }_{Ω} \frac{{γ}_{i}}{{d}_{i}} \cdot exp ( \frac{x}{{d}_{i}} ) \cdot {β}_{k}^{3} ( x ) d x , \\ i = 0 , 1 , 2 , … , 6 and k = 0 , 1 , 2 , … , 554. \qquad Eq . 27$$

Two concrete algorithms to solve eq. 26 for a sparse solution (just N<<K components of t′ are non-zero) of the unknowns t′ are presented in the context of two concrete sample implementations in the following two sections. Once the parameters t′ have been determined, the temperature profile T(x) is calculated as

$$T ( x ) = {\sum }_{k = 0}^{554} \frac{{t}_{k}^{′}}{‖ {a}_{k} ‖} \cdot {β}_{k}^{3} ( x ) . \qquad Eq . 28$$

The resulting reconstructed temperature profile T(x) is finally visualized on a computer screen 5 for visual inspection by the medical doctor or industrial investigator.

In the next two sections, the implementation specific aspects of the presented sample implementations will be presented.

### 1. PC-based Sample Implementation

The sample implementation outlined in FIGS. 6 and 7 is based on an off-the-shelf PC 9. It also comprises a PCI analogue-to-digital converter 15, such as Iotech's DaqBoard\2000 [11] with the provided Matlab [12] drivers, plugged in a PCI-slot 8 of the core PC. This converter converts the analogue signals from the Dicke null-balancing radiometer 7, which represents the voltage encoded brightness temperatures, b, into their digital representations which are directly available inside the Matlab implementation of the reconstruction algorithm. This algorithm implements the general building blocks outlined in FIG. 5, by calculating the matrix A′ through eq. 27, and determining the expansion coefficients t′ by basis pursuit [5]. Basis pursuit solves in the present context the following equation through the interior point algorithm already implemented in Matlab [14, 15]:

min∥t′∥₁, subject to b=A′·t′. Eq. 29

The resulting solution vector t′ represents the expansion coefficients to be used for the temperature profile estimation by eq. 28.

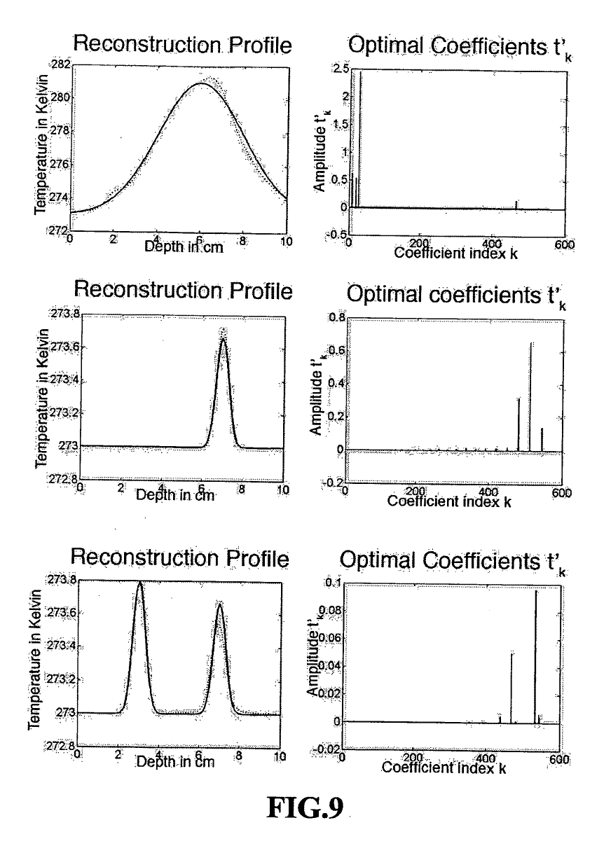

In FIG. 9, three sample results are shown for the presented sample implementation, which used 100 iterations, and a convergence precision of 10⁻³ for the interior point algorithm of the Matlab Optimization Toolbox [12]. The reconstruction results are represented in broken lines and the references in full lines. We see the clear advantage of the disclosed reconstruction approach compared to current state-of-the-art microwave temperature profile reconstruction shown in FIG. 2.

### 2. Embedded Sample Implementation

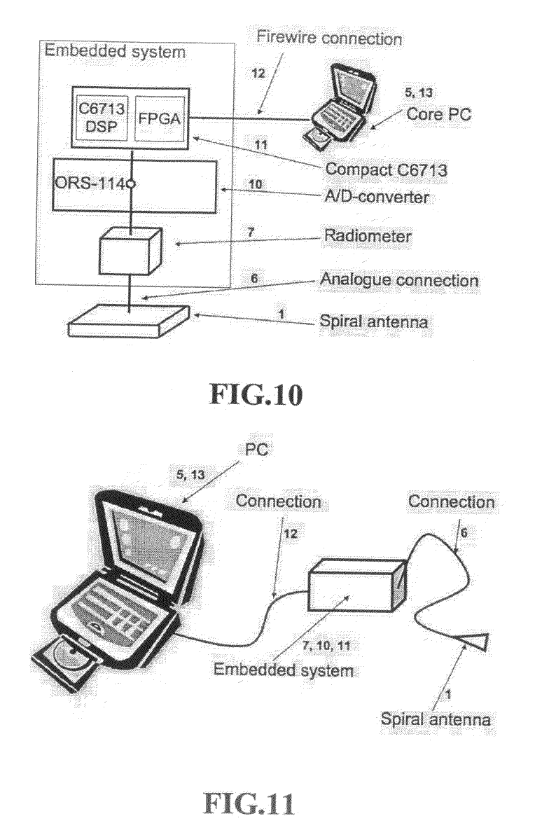

FIG. 10 shows an embedded system for microwave image reconstruction which is connected to a multifrequency spiral antenna 1 via an analogue connection 6 and comprises a dicke null-balancing radiometer 7, an analogue-to-digital converter from Orsys, such as ORS-116, and the embedded development board C6713 Compact from Orsys connected via a firewire connection 12 to a core PC with Unibrain firewire drivers, wherein via said connection 12 images are sent to the core PC and configuration to the embedded system.

The system according to FIG. 11 comprises a PC with firewire drivers and C++ API from Unibrain 5, 13, a microwave spiral antenna 1 and an embedded system 7, 10, 11 for microwave image reconstruction including a Dicke null-balancing radiometer, an analogue-to digital converter from Orsys and a C6713 Compact development board from Orsys. The embedded system 7, 10, 11 is connected to the PC 5, 13 via a firewire connection 12 and via an analogue connection 6 to the antenna 1.

For the embedded sample implementation as outlined in FIGS. 10 and 11, the reconstruction algorithm is implemented on an embedded system 11 from Orsys [21]. More precisely, the radiometer 7 outputs, representing the voltage-encoded brightness temperatures sensed by the multi-frequency spiral antenna, are digitized by the ORS-116 data converter 10 from Orsys [21]. The resulting vector, b, with the brightness temperature components is being employed to reconstruct the temperature profiles on the C6713 Compact 11 from Orsys [21]. The reconstruction result T(x) is transmitted through the FireWire port 12 of the C6713 Compact device to the host computer 13. On the host PC 13, the FireWire drivers by Unibrain [22] are being employed to receive the temperature profiles from the C6713 Compact device 11. Finally, these temperature profiles are getting visualized on the computer screen 5 using the software packages vtk from Kitware [19] and Qt from TroUtech [20].

The reconstruction algorithm implemented on the embedded C6713 Compact device 11 from Orsys [21], calculates the matrix A′ through the mathematical expression given in eq. 27. The FOCUSS algorithm detailed in [18] is used to determine the expansion coefficients t′ for the reconstruction of the temperature profile T(x) through eq. 28. This algorithm solves the following expression iteratively as described in FIG. 12:

min∥b−A′·t′∥₂+λ∥t′∥₁. Eq. 30

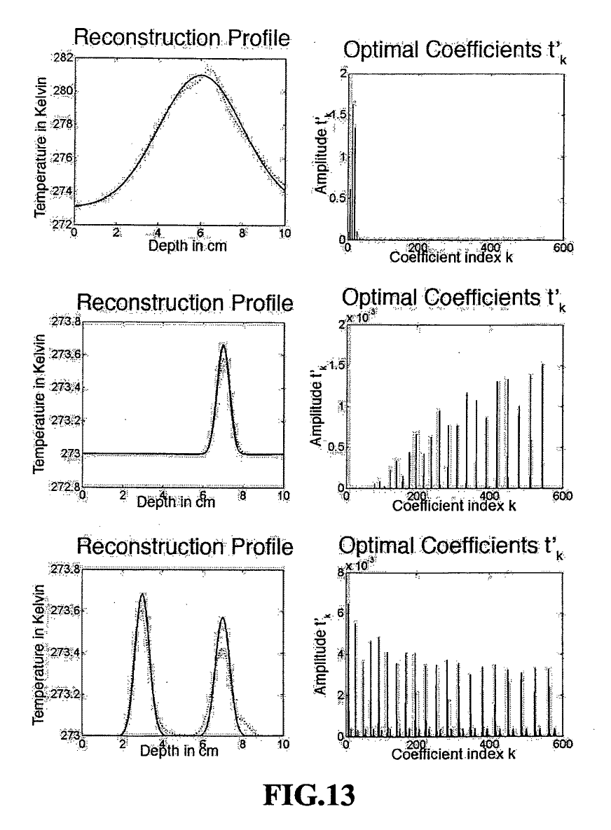

Eq. 30 represents the so-called “basis pursuit de-noising” approach to data decomposition using over-complete dictionaries. In FIG. 13, the reconstruction results for this sample implementation are shown for 500 iterations, λ=0.000001, and where the initial estimate of t′₀ (before any iteration) is a null vector. The reconstruction results are represented in broken lines and the references in full lines. The resulting coefficients t′ determine the temperature profile T(x) by the weighted sum of cubic B-splines as given in eq. 28.

### REFERENCES

[1] B. Stec, A. Dobrowolski, and W. Susek: “Multifrequency microwave thermography for biomedical applications” IEEE Transactions on Biomedical Engineering, Vol 51(3), March 2004
[2] S. Jacobsen and P. R. Stauffer: “Nonparametric 1-D temperature restoration in lossy media using Tikhonov regularization on sparse radiometry data”, IEEE Transactions on Biomedical Engineering, Vol. 50(2), February 2003
[3] S. Mallat and Z. Zhang: “Matching pursuit with time-frequency dictionaries”, IEEE Transactions on Signal Processing, Vol. 41, December 1993
[4] Y. C. Pati, R. Rezaiifar, and P. S. Krishnaprasad: “Orthogonal matching pursuits: recursive function approximation with applications to wavelet decomposition”, Proceedings of the 27^{th} Asilomar Conference in Signals, Systems, and Computers, 1993
[5] S. S. Chen, D. L. Donoho, and M. A. Saunders: “Atomic decomposition by basis pursuit”, SIAM J. Sci. Comput., Vol 20(1), 1998
[6] S. Jaggi, W. C. Karl, St. Mallat, and A. S. Willsky: “High resolution pursuit for feature extraction”, Technical Report, MIT, November 1996
[7] L. M. Delves, and J. L. Mohamed: “Computational methods for integral equations”, Cambridge University Press, 1985
[8] W. H. Press, S. A. Teukolsky, W. T. Vetterling, and B. P. Flannery: “Numerical Recipes in C”, Cambridge University Press, 1992 (Second Edition)
[9] S. Jacobsen and P. R. Stauffer “Multifrequency Radiometric Determination of Temperature Profiles in a Lossy Homogeneous Phantom Using a Dual-Mode Antenna with Integral Water Bolus”, IEEE Transactions on Microwave Theory and Techniques, vol. 50(7), pp. 1737-1746, July 2002.
[10] S. Jacobsen, P. R. Stauffer, and D. Neuman: “Dual-mode Antenna Design for Microwave Heating and Noninvasive Thermometry of Superficial Tissue Disease”, IEEE Transactions on Biomedical Engineering, vol. 47, pp. 1500-1509, November 2000.
[11] http:/www.iotechcom
[12] http://www.matlab.com
[13] G. B. Dantzig, A. Orden, and P. Wolfe: “Generalized Simplex Method for Minimizing a Linear from Under Linear Inequality Constraints”, Pacific Journal Math, vol. 5, pp. 183-195.
[14] S. Mehrotra: “On the implementation of a Primal-Dual Interior Point Method”, SIAM Journal on Optimization, vol. 2, pp. 575-601, 1992.
[15] Y. Zhang: “Solving Large-Scale Linear Programs by Interior-Point Methods under the MATLAB Environment”, Technical Report TR96-01, Department of Mathematics and Statistics, University of Maryland, Baltimore County, Baltimore, Md., July 1995
[16] T. F. Coleman, and Y. Li: “A Reflective Newton Method for Minimizing a Quadratic Function Subject to Bounds on some of the Variables”, SIAM Journal on Optimization, vol. 6(4), pp. 1040-1058, 1996.
[17] P. E. Gill, W. Murray, and M. H. Wright: “Practical Optimization” Academic Press, London, UK, 1981.
[18] B. D. Rao, K Engan, S. F. Cotter, J. Palmer, and K Kreutz-Delgado: “Subset Selection in Noise Based on Diversity Measure Minimization”, IEEE Transactions on Signal Processing, vol 51(3), pp. 760-770, March 2003
[19] http://www.vtk.org
[20] http://www.trolltech.com
[21] http://www.orsys.de
[22] http://www.unibrain.com
[23] I. N. Bronstrein, K. A. Semendjajew, G. Musiol, and H. Mühlig “Taschenbuch der Mathematik”
   Verlag Harri Deutsch, 2^{nd} edition, 1995

1. A microwave temperature image reconstruction system, which reconstructs from passively sensed microwave data temperature profiles or maps by means of a known optimization algorithm for solving sparse data decomposition, including one or several multi-frequency microwave antennas for sensing the brightness temperatures, a radiometer for interpreting the signal and translate the sensed microwave radiation power into units of voltage, an analogue-to-digital converter for digitizing said analogue voltage, a reconstruction unit for reconstructing a temperature profile which caused the sensed microwave radiation, and means for visualizing said temperature profiles, wherein the microwave image reconstruction is carried out by solving the following integral equations:
$${b}_{i} = {γ}_{i} {\int }_{Ω} {W}_{i} ( x ) \cdot T ( x ) d x , \\ with \\ i = 1 , 2 , … , M \qquad Eq . A$$
where bᵢ is the measured brightness temperature sensed by the imaging antenna at frequency fᵢ, γᵢ is the antenna efficiency at frequency fᵢ, Wᵢ(x) is the spatial weighting function at frequency fᵢ, Ω is the sensing space of the microwave antenna, and T(x) is the spatial temperature distribution the system aims to recover, where i indexes the M discrete measurement frequencies fᵢ of the system, and where the weighting function Wᵢ(x) weights the brightness temperature contributions from different spatial positions x, and
wherein in order to reconstruct one-, two- or three-dimensional temperature profiles or maps of an intra-body temperature profile which caused said sensed microwave radiation,
said system comprises the use of over-complete dictionaries, the use of weighting functions Wi(x) at the measurement frequencies fᵢ which are obtained from numerical simulations with an electromagnetic simulator so that the functions Wᵢ(x) are given as discrete functions over the measuring space Ω, and the use of an integration of the equations which is performed numerically.

2. System according to claim 1, wherein an approach to solve equation A for the spatial temperature distribution T(x) includes the following step:
construct a function sub-space Ω_{D} of the space of square-integratable functions L₂, spanned at least approximately by an over-complete set of a first number K of functions, D={hₖ(x)}, k=1, 2, . . . , K, which accounts for the variability of possible temperature profiles T(x), this set being constructed in a way that for any temperature profile T(x), which can be expected, a weighted sum of functions of D exists, which approximates the profile T(x):
$$T ( x ) \approx {\sum }_{k = 1}^{K} {t}_{k} \cdot {h}_{k} ( x ) , \qquad Eq . B$$
with just a second number N of coefficients tₖ being non-zero, and said second number N being much smaller than said first number K.

3. System according to claim 1, including the following step:
once such a set D has been constructed, for any given set of measurements bᵢ said optimization reconstruction algorithm determines both, the functions of D and its non-zero weighting coefficients tₖ which approximate the optimized temperature profile T(x) by the weighted sum given in said equation B.

4. System according to claim 1, wherein the equation A is reformulated as follows:
$${b}_{i} = {\sum }_{k = 1}^{K} {t}_{k} \cdot {γ}_{i} \cdot {\int }_{Ω} {W}_{i} ( x ) \cdot {h}_{k} ( x ) d x . \qquad Eq . C$$
written in matrix form as:
b=A·t, Eq. D
with
$${A}_{i , k} = {γ}_{i} \cdot {\int }_{Ω} {W}_{i} ( x ) \cdot {h}_{k} ( x ) d x . \qquad Eq . E$$
wherein the size of the matrix A is given by the number of measurements M of the system and by the number of functions K in the over-complete set D, so that the number of rows is M while the number of columns is K, wherein equation E is considered as a transformation F which transforms the space Ω_{D} spanned by the basis functions of the set D onto a subspace of R^{N}, denoted Ω_{R}, where R^{N} is the vector space of N-dimensional vectors of real numbers:
$$F \text{:} {Ω}_{D} ⋐ {L}_{2} -> {Ω}_{R} ⊆ {R}^{N} , \\ {h}_{k} ( x ) -> {a}_{k} = {\int }_{Ω} W ( x ) \cdot {h}_{k} ( x ) d x , \\ k = 1 , 2 , … k , \\ with \\ W ( x ) := ( \begin{matrix}\begin{matrix}\begin{matrix}{γ}_{1} \cdot {W}_{1} ( x ) \\ {γ}_{2} \cdot {W}_{2} ( x )\end{matrix} \\ …\end{matrix} \\ {γ}_{M} \cdot {W}_{m} ( x )\end{matrix} ) \qquad Eq . F$$
wherein the resulting subspace Ω_{R} of R^{N} is considered as a vector space spanned by the vectors {aₖ} which are just the rows in the matrix A of equation D, and including the step of normalizing the vectors aₖ and therefore the rows A, the temperature profile reconstruction of equation D can be reformulated as standard data decomposition with over-complete dictionaries as follows:
$$b = {\sum }_{k = 1}^{K} {t}_{k}^{′} \cdot {a}_{k}^{′} , \\ with \\ {t}_{k}^{′} = {t}_{k} \cdot ‖ {a}_{k } ‖ , \\ and \\ {a}_{k}^{′} = \frac{{a}_{k}}{‖ {a}_{k} ‖} . \qquad Eq . G$$
where the symbol ∥aₖ∥ stands for the classical vector norm of R^{N} defined by
$$‖ {a}_{k} ‖ = \sqrt{{\sum }_{i} {a}_{i}^{2}} ,$$
and wherein therefore by solving equation G for a sparse solution of the coefficients t′ₖ the expansion coefficients for the reconstruction of the temperature profile T(x) through equation B are obtained.

5. System according to claim 1, having a reliable profile reconstruction approach applicable to industrial, medical, metrological or radiometric temperature measurement or monitoring applications.

6. System according to claim 1, comprising an over-complete dictionary of a first number K of basis functions, such as B-splines at different scales and positions, in order to represent the variety of possible real-world temperature profiles with a second number N of these basis functions, and a regularized reconstruction algorithm which selects the optimal or optimized set of N basis functions out of the over-complete set of K basis functions and which determines their weights to reconstruct the temperature profile, and wherein said second number N is smaller than said first number K.

7. System according to one claim 4, wherein for solving equation G at least one of the following algorithms are used: matching pursuit, orthogonal matching pursuit, basis pursuit and/or high-resolution matching pursuit, or wherein algorithms are used to solve said equation G which optimize an equation consisting of two parts, first the data part, which considers the actual measurements to determine the optimal or optimized temperature profile T(x) according to said coefficients t′ₖ, and second the regularization part, which ensures that just few of the coefficients t′ₖ are different from zero.

8. System according to claim 1, wherein said radiometer is a null-balancing radiometer and further comprising at least one multi-frequency spiral microwave antenna, wherein the signal sensed by the antenna passes through an analogue connection to said null-balancing radiometer which detects sequentially the signal contributions at the different frequencies of the multi-frequency antenna of the system, the resulting sequential analogue signals from the radiometer being directly related to the brightness temperatures at the corresponding frequencies, and these brightness temperatures being directly related to a real intra-body temperature distribution, and comprising means to reconstruct a one dimensional profile of real intra-body temperatures from the brightness temperatures via the outputs from the analogue-to-digital converter.

9. System according to claim 2, comprising means for specifying an over-complete set of basis functions {hₖ(x)} of Ω_{D} and an optimization algorithm for one-dimensional temperature profile reconstruction having a spatial variable x, wherein said variable parameterizes a one-, two- or three-dimensional imaging space Ω and wherein the size of the imaging space Ω is given by the maximal sensing depth dₘₐₓ of the microwave antenna and the antenna or antennas bandwidths.

10. System according to claim 2, where the basis functions {hₖ(x)} build an over-complete set of basis functions of Ω_{D}, i.e. the basis functions are chosen, so that for any function f(x) in Ω_{D}, L>1 sets of coefficients {t_{k,i}}, J, i=1, 2, . . . , L exist for which
$$f ( x ) \approx {\sum }_{k = 1}^{K} {t}_{k , i} \cdot h ( x ) , \\ i = 1 , 2 , … , L . \qquad Eq . H$$
wherein the said basis functions {hₖ(x)} are related to the existence of one set of coefficients {tₖ} out of the sets {t_{k,i}}, i=1, 2, . . . , L which fulfills equation H and for which just N of the K coefficients with N<<K is non-zero.

11. System according to claim 6, wherein B-splines of order N_{d} and at Nₛ consecutive scales are used to build the over-complete set D of basis functions, wherein at the lowest scale 4 B-splines cover the whole reconstruction space Ω, while at each higher scale, one spline is added, so that at each scale s a number of Nₚ₍ₛ₎=s+4 B-splines at different positions are added to said set D, and wherein the whole over-complete set of basis functions is parameterized as:
D={hₖ(x)}={β_{s,p(s)}³(x)}, Eq. J
s=0, 1, . . . , Nₛ,
p(s)=1, 2, . . . , s+4.
wherein β³ is given for the order N_{d}=3, but wherein D may be in general applied for the order N_{d}≠3.

12. System according to claim 11, with
$${β}_{s , p ( s )}^{3} ( x ) = \{ \begin{matrix}\begin{matrix}\frac{2}{3} - {\vert x \vert}^{2} + \frac{{\vert x \vert}^{3}}{2} , if 0 \leq \vert ( \begin{matrix}x - {d}_{m ax} \cdot \\ \frac{( p ( s ) - 2 )}{{N}_{p ( s ) } - 3}\end{matrix} ) \cdot \frac{{N}_{p ( s )} - 3}{{d}_{m ax}} \vert \leq 1 , \\ \frac{{( 2 - \vert x \vert )}^{3}}{6} , if 1 \leq \vert ( \begin{matrix}x - {d}_{m ax} \cdot \\ \frac{( p ( s ) - 2 )}{{N}_{p ( s )} - 3}\end{matrix} ) \cdot \frac{{N}_{p ( s )} - 3}{{d}_{ma x}} \vert \leq 2 ,\end{matrix} \\ 0 , otherwise .\end{matrix} \qquad Eq . K$$
for the order N_{d}=3

13. System according to claim 4, comprising an off-the-shelf PC, a PCI analogue-to-digital converter, preferable plugged in a PCI-slot of the core PC, wherein said converter converts the analogue signals from a null-balancing radiometer, which represents the voltage encoded brightness temperatures (b) into their digital representations which are directly available inside the implementation of the reconstruction algorithm, wherein for the transformation integral of equation F the following recursive formula is used
$$\int {x}^{n} \cdot {e}^{ax} d x = \frac{1}{a} \cdot {x}^{n} \cdot {e}^{ax} - \frac{n}{a} \int {x}^{n - 1 } \cdot {e}^{ax} d x . \qquad Eq . L$$
and wherein the expansion coefficients t′ of equation L are determined by basis pursuit, wherein said basis pursuit solves the following equation through the interior point algorithm:
min∥t′∥₁, subject to b=A′·t′. Eq. M

14. System according to claim 4, including an embedded sample implementation calculating the Matrix A′ through the mathematical expression given in the following equation
min∥t′∥₁, subject to b=A′·t′. Eq. M
and solving the following expression iteratively
min∥b−A′·t′∥₂+λ·∥t′∥₁. Eq. N

15. System according to claim 1, comprising one single one-dimensional multi-frequency spiral microwave antenna operating at 7 frequencies in the range of 0.5 to 3.75 GHz, wherein the signal sensed by the antenna passes through an analogue connection to a Dicke null-balancing radiometer which detects sequentially the signal contributions at the different frequencies of the multi-frequency antenna of the system.
